SEQUENCE LISTING (2 sequences)

SEQ ID NO: 1 - protein, 118 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVR

SEQ ID NO: 2 - protein, 221 aa, Homo sapiens
EPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVR

(19) United States
(12) Patent Application Publication
(10) Pub. No.: US 2022/0105153 A1
(43) Pub. Date: Apr. 7, 2022

(54) USE OF PROTEIN-BASED LONG-ACTING PREPARATION IN IMPROVING SEXUAL DYSFUNCTION

(71) Applicant: Xintrum Pharmaceuticals, Ltd., Nanjing (CN)

(72) Inventors: Yi Zhang, Nanjing (CN); Hai Chen, Nanjing (CN); Gaoyong Liao, Nanjing (CN)

(21) Appl. No.: 17/555,374

(22) Filed: Dec. 18, 2021

Related U.S. Application Data
(63) Continuation of application No. PCT/CN2020/108912, filed on Aug. 13, 2020.

(30) Foreign Application Priority Data
Aug. 29, 2019 (CN) 201910809334.6

(51) Int. Cl. A61K 38/17; A61P 15/10

(52) U.S. Cl. CPC A61K 38/1709; A61P 15/10

(57) ABSTRACT

A long-acting preparation made from a protein having the amino acid sequence of SEQ ID NO. 1 or SEQ ID NO. 2 or from a modified protein of the protein for improving sexual dysfunction. The modified protein is the protein molecule connected with another protein or with another chemical substance.

### TECHNICAL FIELD

The present invention relates to a use of a protein-based long-acting preparation in improving sexual dysfunction.

### DESCRIPTION OF RELATED ART

Erectile dysfunction (hereinafter referred to as ED for abbreviation) is the most common male sexual dysfunction. Its etiology can be divided into: psycho mental ED, organic ed (including vascular and neurological causes), or mixed ED (referring to erectile dysfunction caused by psycho mental factors and organic causes).

Existing chemical drugs for treating ED, such as sildenafil citrate, can only restore an abnormal erectile function to a normal state temporarily but cannot treat the organic lesion that causes ED.

As ED has a high incidence rate and is a chronical disease, it is imperative to develop new, long-acting drugs that can improve ED by reversing the underlying organic lesion.

Because the incidence of ED is high and it is a chronic disease, it is very necessary to develop new long-acting drugs that can provide the treatment to ED by reversing the organic lesions.

### BRIEF SUMMARY OF THE INVENTION

One objective of the present invention is to find a long-acting preparation that can improve ED.

The inventor of the present invention has found through experimentation that a long-acting preparation made from a protein having the amino acid sequence of SEQ ID NO. 1 or SEQ ID NO. 2 can improve ED in experimental animals.

The long-acting preparation disclosed herein includes various sustained-release preparations or controlled-release preparations made from the aforesaid protein and a pharmaceutically acceptable auxiliary substance.

The long-acting preparation disclosed herein also includes long-acting preparations made from a modified protein of the aforesaid protein.

The aforesaid modified protein is a protein molecule having the amino acid sequence of SEQ ID NO. 1 or SEQ ID NO. 2 and connected with another protein or with another chemical substance.

Experimental results have proved that the protein-based long-acting preparation provided by the present invention can effectively improve ED, as detailed below with reference to an embodiment of the invention.

### BRIEF DESCRIPTION OF THE SEVERAL VIEWS OF THE DRAWINGS

FIG. 1 shows the latent periods of catch action and the numbers of times of catch action of experimental rats, wherein:
(a) shows the latent periods of catch action of the experimental rats, and
(b) shows the numbers of times of catch action of the experimental rats;

FIG. 2 shows the erectile function test results of the rats, wherein the erectile function is indicated by the intracavernous pressure of the rats in each group: (a) blank control group; (b) diabetic model control group; (c) ZX1305 low dose group; (d) ZX1305 high dose group.

FIG. 3 shows an assessment of the erectile function of the experimental rats in different groups, wherein the erectile function are expressed as ratios of the intracavernous pressure to the average arterial pressure.

FIG. 4 Testicular index of experimental rats in each experimental group.

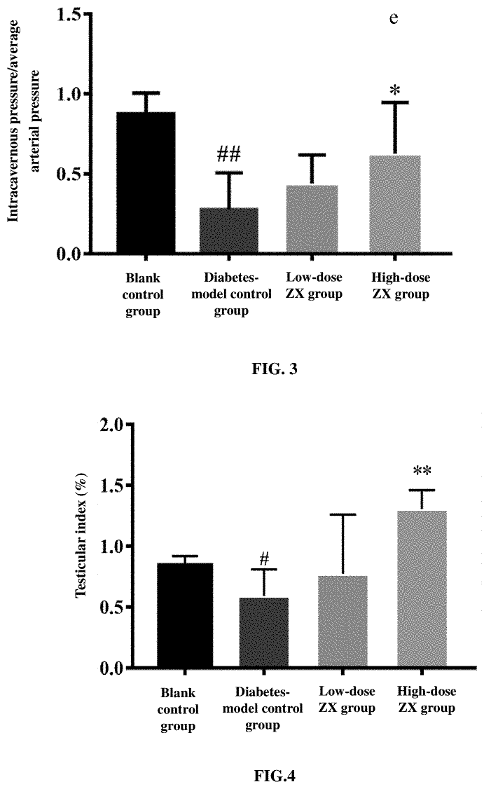

Explanation: In FIG. 3 and FIG. 4, “blank control group” refers to the “normal-animal control group” in the following text, and “diabetes-model control group” refers to the “diabetes-model blank control group” in the following text; and

ZX indicates the experimental group using a protein having the amino acid sequence of SEQ ID NO. 1.

### DETAILED DESCRIPTION OF THE INVENTION

In the following embodiment, “ZX1305 protein” indicates a protein having the amino acid sequence of SEQ ID NO. 1.

Embodiment 1: Experimental Study on Rat ED Improvement by ZX1305-Protein-Based Sustained-Release Preparation

### 1. Experimental Animals, Experimental Materials, and Instruments

### 1.1 Experimental Animals

40 specific-pathogen-free (SPF) male Sprague Dawley (SD) rats provided by Qing Long Shan Animal Propagation Station, Jiangning District, Nanjing City

The 40 male SD rats received adaptive feeding for three days. After that, ten rats were randomly selected as the normal-animal blank control group and fed with normal feed. The remaining 30 rats were fed with high-fat feed in order to create a diabetes model.

In the fourth week, the rats in the normal-animal blank control group were given a citric acid-sodium citrate buffer solution for comparison purposes.

Meanwhile, the rats fed with high-fat feed were intraperitoneally injected with a streptozotocin (STZ) solution (with a dose of 60 mg/kg, and the solvent being a citric acid-sodium citrate buffer solution), and the drug was administered for three consecutive days.

The blood sugar level was tested on the fourth day after drug administration. The intended diabetes model would be considered established if a random plasma glucose level higher than 16.7 mmol/L was detected.

The effects of the ZX1305 sustained-release preparation on the body weight and blood sugar level of the rats are tabulated as Table 1.

TABLE 1
Body weights and blood sugar levels of the experimental rats
in the diabetes-model groups (mean ± standard deviation)

| | Number of | Body weight (g) | | Blood sugar (mM) | |
|---|---|---|---|---|---|
| Group | rats | Week 1 | Week 10 | Week 4 | Week 10 |
| Normal-animal blank | 10 | 196.8 ± 7.3 | 354.9 ± 36.5 | 6.0 ± 1.1 | 7.3 ± 0.6 |
| control group | | | | | |
| Diabetes-model blank | 8 | 196.2 ± 12.1 | 167.0 ± 19.2## | 6.3 ± 0.9 | 26.0 ± 1.9## |
| control group | | | | | |
| Low-dose ZX1305 | 9 | 205.6 ± 17.5 | 163.9 ± 20.2## | 6.3 ± 1.2 | 34.0 ± 4.6## |
| group | | | | | |
| High-dose ZX1305 | 8 | 200.3 ± 14.9 | 180.1 ± 25.7## | 6.5 ± 1.7 | 32.1 ± 4.8## |
| group | | | | | |

##P < 0.01, compared with the normal-animal blank control group.

Six weeks after the injection of STZ, a comparison with the normal-animal blank control groups shows that the rats in the diabetes-model blank control group, in the high-dose ZX1305 group, and in the low-dose ZX1305 group all had a significant decrease in body weight (P<0.01) and a significant increase in blood sugar level (P<0.01). This indicates that the diabetic rat model was successfully established.

### 1.2 Instruments

### Digital Camera, Multi-Channel Electrophysiological Recording System, Pressure Converter, Stimulating Electrodes, Computer, and Plate Reader

### 1.3 Drugs/Major Reagents

Streptozotocin (STZ) repackaged by sigma, purchased from ThermoFisher Biochemical Reagent Co., Nanjing

ZX1305 sustained-release preparation provided by XINTRUM Pharmaceuticals, Jiangsu. The preparation is a nanoparticle long-acting preparation prepared by the methods disclosed in PCT/US2019/015, filed on Jan. 29, 2019, and U.S. Provisional Patent Application No. 62/623,018, filed on Jan. 29, 2018.

### 1.4 Preparation of the Drugs to be Tested

\textcircled{1} Citric acid-sodium citrate buffer solution: 2.1 g of citric acid (FW: 210.4) was weighed and then dissolved in 100 ml of double distilled water to produce solution A. 2.94 g of sodium citrate (FW: 294.10) was dissolved in 100 ml of double distilled water to produce solution B. Solutions A and B were mixed at a ratio of 1:1, and the pH value of the mixture was adjusted to 4.2-4.5 with a solvent or water. The buffer solution was used immediately after the preparation.

\textcircled{2} Solutions of the ZX1305 sustained-release preparation: The specification of the sustained-release preparation was 0.5 mg/piece. Immediately before use, an entire vial of the sustained-release preparation/drug was directly dissolved in 8 ml of ZX1305 preparation buffer solution to produce the high-dose drug solution (62.5 μg/ml) to be administered to the high-dose group. The high-dose drug solution was then diluted ten times with the ZX1305 preparation buffer solution to produce the low-dose drug solution (6.25 μg/ml) to be administered to the low-dose group.

### 2. Experimental Method

### 2.1 Drug Administration to the Experimental Animals by Groups

The rats in the successfully established diabetes model were randomly divided into three groups:

the diabetes-model blank control group, the low-dose (10 μg/kg) ZX1305 sustained-release preparation group, and the high-dose (100 μg/kg) ZX1305 sustained-release preparation group. These three groups and the normal-animal blank control group made up a total of four groups.

The drug-administered groups were intramuscularly injected with their respective ZX1305 sustained-release preparation solutions. The volume of the drugs administered was 1.6 ml/kg, and the drugs were administration only once. After drug administration, the behaviors of the rats were observed on a daily basis, changes in body weight were recorded on a weekly basis, and the blood sugar level was recorded every two weeks. The observation and recording continued for six weeks.

### 2.2 APO Induction Experiment

An APO induction experiment was conducted in the tenth week after drug administration. Each rat was subcutaneously injected in the neck with apomorphine (APO) at 100 μg/kg and was observed for 30 minutes after the injection, with the occurrence or non-occurrence of penile erection and the number of times of penile erection recorded. The erection rates were then calculated. Glans engorgement plus exposure of the terminal end of the penis body was counted as one penile erection.

### 2.3 Observation of Sexual Behaviors

Rat cages were prepared, in each of which a single male experimental rat was placed. The cages were placed in the dark for five minutes, allowing the rats to adapt to the environment. Then, a normal female rat was put into each cage, and the following began to be recorded: ^{\textcircled{1}} the latent capture period: the time period starting from the instant a female rat was put into a cage to the instant the male rat in the cage captured the female rat for the first time (the latent capture period was recorded as 20 minutes if the male rat did not capture the female rat at all); ^{\textcircled{2}} the number of times of capture: the number of times for which a male rat captured the female rat in its cage during the 20 minutes after the female rat was put into the cage (the number of times of capture was recorded as 0 if the male rat did not capture the female rat at all).

### 2.4 Determination of the Intracavernous Pressure

The cavernous pressure of each rat was recorded with the electrophysiological recording system while the cavernous nerves of the rat were stimulated with a 5-V, 15-Hz, 5-ms direct current.

### 2.5 Determination of the Average Arterial Pressure

Once the intracavernous pressure of a rat was determined, the abdominal aorta of the rat was exposed, and a PE50 tube connected to the pressure transducer was placed into the aorta in order to monitor the average arterial pressure of the rat directly and continuously.

### 2.6 Calculation of the Testicular Index

After the rats in each group were weighed and anesthetized, whole blood was drawn from the abdominal aorta and then centrifuged at 4° C. and 2000 g for ten minutes. Serum was subsequently obtained and kept at −20° C. for later use. The rats were then executed by cervical dislocation. The testicles of the rats were taken and weighed with precision in order to calculate the testicular index as follows: testicular index (%)=testicle weight (g)/body weight (g)×100%.

### 2.7 Extraction of Other Tissues

Once the rats were executed, the heart, liver, spleen, lungs, kidneys, brain, eyeballs, cavernous body, and sciatic nerves of each rat were extracted.

### 3. Experimental Results

### 3.1 The APO Induction Experiment

Observation of the ten rats in the normal-animal blank control group reveals that every rat had penile erection; the erection rate was 100%, and the average number of times of erection was 5.3. Observation of the eight rats in the diabetes-model blank control group reveals that none but one of the rats had penile erection, the erection rate being 12.5%. The results indicate that a pathological model for sexual dysfunction of diabetic rats was successfully established.

According to the blood sugar levels of the rats and the results of the APO induction experiment, a sexual dysfunction model for diabetic rats was successfully established.

### 3.2 The Effect of the ZX1305 Sustained-Release Preparation on the Sexual Behaviors of the Rats

The latent capture periods and the numbers of times of capture of the experimental rats are shown in Table 2 and FIG. 1.

TABLE 2
Latent capture periods and numbers of times of capture
of the experimental rats (mean ± standard deviation)

| | Number or | Latent capture | Number of times |
|---|---|---|---|
| Group | rats | period (sec) | of capture |
| Normal-animal | 4 | 80.3 ± 12.7 | 18.5 ± 2.6 |
| blank control group | | | |
| Diabetes-model | 4 | 1039.5 ± 321.0## | 0.3 ± 0.5## |
| blank control group | | | |
| Low-dose ZX1305 | 4 | 400.5 ± 34.9** | 1.5 ± 1.0 |
| group | | | |
| High-dose ZX1305 | 4 | 280.8 ± 22.3** | 4.5 ± 1.3** |
| group | | | |

##P < 0.01, compared with the normal-animal blank control group;
**P < 0.01, compared with the diabetes-model blank control group.

Compared with the normal-animal blank control group, the diabetes-model blank control group showed a significant increase in the latent capture period (P<0.01) and a significant reduction in the number of times of capture (P<0.01).

Compared with the diabetes-model blank control group, the high-dose ZX1305 group showed a significant improvement in both the latent capture time and the number of times of capture (P<0.01), and the low-dose ZX1305 group showed a significant reduction in the latent capture period (P<0.01) but no significant change in the number of times of capture (P>0.05).

### 3.3 The Effect of the ZX1305 Sustained-Release Preparation on the Erectile Function of the Rats

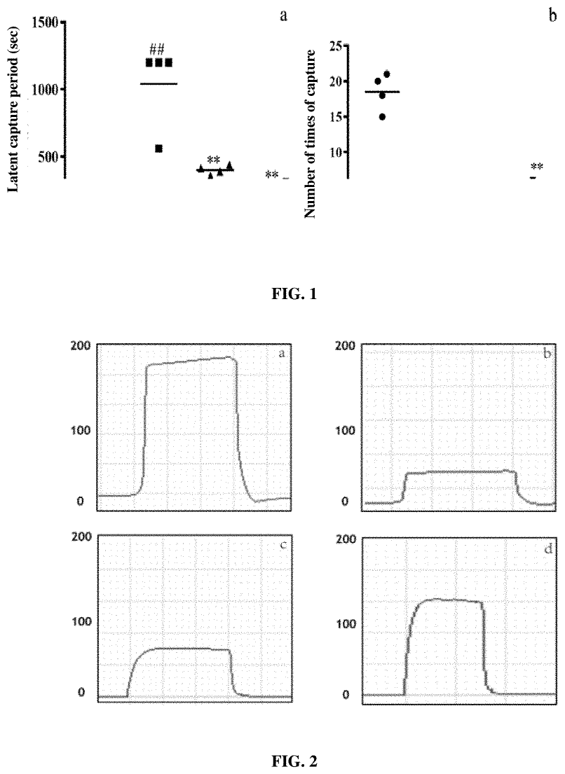

The test results are shown in FIG. 2 and FIG. 3.

FIG. 2 shows the erectile function test results of the rats, wherein the test results are expressed as ratios of the intracavernous pressure to the average arterial pressure.

FIG. 3 shows an assessment of the erectile function of the experimental rats in different groups. More specifically, FIG. 3 is a chart in which the erectile function is indicated by the intracavernous pressure of the rats in each group.

##P<0.01, compared with the normal-animal blank control group; *P<0.05, compared with the diabetes-model blank control group.

Compared with the normal-animal blank control group, the diabetes-model blank control group showed a highly significant reduction in the ratio of the intracavernous pressure to the average arterial pressure (P<0.01).

Compared with the diabetes-model blank control group, the high-dose ZX1305 group showed a significant increase in the ratio of the intracavernous pressure to the average arterial pressure (P<0.01).

While the low-dose ZX1305 group also showed an increase in the ratio of the intracavernous pressure to the average arterial pressure, the change is not significant (P>0.05).

### 3.4 The Effect of the ZX1305 Sustained-Release Preparation on the Testicular Indices of the Rats

#P<0.05, compared with the normal-animal blank control group; **P<0.01, compared with the diabetes-model blank control group.

Compared with the normal-animal blank control group, the diabetes-model blank control group showed a significant reduction in the testicular index (P<0.05).

Compared with the diabetes-model blank control group, the high-dose ZX1305 group showed a significant increase in the testicular index (P<0.01), and the low-dose ZX1305 group showed no significant change in this respect (P>0.05). The experiment results are plotted in FIG. 4.

The foregoing experiments have shown that:

1) When administered at a dose of 100 μg/kg, the ZX1305 sustained-release preparation produced the following experimental results on the diabetic rats with sexual dysfunction:

^{\textcircled{1}} A highly significant increase in the number of times of capture;

^{\textcircled{2}} A highly significant increase in the testicular index;

^{\textcircled{3}} A significant increase in the ratio of the intracavernous pressure to the average arterial pressure; and

^{\textcircled{4}} A highly significant reduction in the latent capture period.

2) When administered at a dose of 10 μg/kg, the ZX1305 sustained-release preparation caused a highly significant reduction in the latent capture period of the diabetic rats with sexual dysfunction but did not have a significant effect on the other indices.

3) The ZX1305 sustained-release preparation had no effect on the body weights or blood sugar levels of the diabetic rats.

### 4. Conclusion of the Experiments

The ZX1305-protein-based sustained-release preparation had an improving effect on the sexual dysfunction of the diabetic rats.

1. A method of improving sexual dysfunction of a subject, comprising: administering to the subject a long-acting preparation comprising a protein comprising the amino acid sequence of SEQ ID NO. 1 or SEQ ID NO. 2.

2. The method of claim 1, wherein the sexual dysfunction is erectile dysfunction (ED).

3. The method of claim 1, wherein the long-acting preparation are sustained-release preparation or a controlled-release preparation comprising said protein and a pharmaceutically acceptable excipient.

4. The method of claim 1, wherein the protein comprises a modified protein thereof.

5. The method of claim 4, wherein the modified protein is the protein molecule connected with other protein or with other chemical substance.

6. A long-acting preparation for improving sexual dysfunction, comprising a protein having the amino acid sequence of SEQ ID NO. 1 or SEQ ID NO. 2, or a modified protein thereof.

7. The long-acting preparation of claim 6, wherein the modified protein is the protein molecule connected with another protein or with another chemical substance.

8. The long-acting preparation of claim 6 or 7, which is a sustained-release preparation or a controlled-release preparation.

9. The long-acting preparation of claim 8, which is a nanoparticles long-acting preparation comprising a protein having the amino acid sequence of SEQ ID NO. 1.
